(19) United States
(12) Patent Application Publication
(10) Pub. No.: US 2006/0264346 A1
(43) Pub. Date: Nov. 23, 2006

(54) Timed-release cleansing and/or treatment formulation and method for making and using the same

(76) Inventor: Mary Katherine Sullivan, West Seneca, NY (US)

Correspondence Address: PATRICIA M. COSTANZO;PATS PENDING, P.O. BOX 101, ELMA, NY, 14059, US

(21) Appl. No.: 11/132,574

(22) Filed: May 19, 2005

(51) Int. Cl. A61K 8/00

(52) U.S. Cl. 510/137

(57) ABSTRACT

A low cost, easy to use cleanser and/or treatment formulation (available in a powder, liquid, or solid form) includes encapsulated, non-toxic, timed-release dye. The dye remains encapsulated during cleansing and/or treating, such as during hand washing for the recommended 15-20 seconds. The encapsulant then releases the dye indicating that the hands have been thoroughly washed. Once the dye/lather mixture is completely removed by rinsing, the hand washer is assured that his or her hands have been thoroughly washed and rinsed completely. In those cases where an anti-bacterial agent is recommended, it should be used for the recommended 15-20 seconds, in order to be effective. The invention may be formulated for any type of timed cleaning and/or treatment, including hair washing, pet cleaning, hair coloring, and so forth. The cleansing formulation may be aqueous or organic based and may include encapsulated scents, lotions, medicants, therapeutic agents, and/or anti-microbials.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE OR A COMPUTER PROGRAM LISTING COMPACT DISK APPENDIX

Not Applicable

### BACKGROUND

The present invention relates generally to cleansing formulations and, more particularly, to a formulation that indicates when cleansing is complete, and yet more particularly to a hand-washing cleansing formulation that comprises a timed-release encapsulated dye that is released when hands have been washed long enough to indicate effective removal of infectious agents and dirt.

The background information discussed below is presented to better illustrate the novelty and usefulness of the present invention. This background information is not admitted prior art.

Most infectious diseases, such as the common cold rhinovirus, food borne illnesses such as those caused by certain strains of E. coli, salmonella, and other bacteria, and gastrointestinal infectious agents such as the rotavirus are spread by person-to-person contact or from people touching surfaces contaminated by these infectious agents. Surfaces become contaminated in many ways, such as when people with a communicable illness rub their noses and/or eyes and then touch a surface. Likewise, when people who are sick use their hands to stifle a cough, their hands become a vector for the spread of disease.

Many people are unaware that infectious agents are able to live on hard, dry surfaces for up to several hours. In fact, rotavirus, which is an infectious agent that causes gastrointestinal illness, can remain infectious on a dry, smooth surface for as long as 20 minutes, while certain strains of E. coli, salmonella, and other bacteria can live on surfaces like cafeteria tables and door-knobs for up to two hours. Some of the other surfaces that we contact perhaps many times each day that are known to harbor infectious agents include light switches, bath and kitchen faucets, telephone receivers, toilet handles, remote control devices, refrigerator handles, computer keys, microwaves, kitchen drains, toilet seat tops, kitchen dishcloths, toddler's toys, kitchen counters, diaper areas, outdoors seating devices, such as park benches, and grab bars on public transportation vehicles.

Ordinary diseases, such as those mentioned above, may result in many different kinds of societal losses. For example, nearly 22 million school days are lost annually due to the 52.2 million cases of the common cold that affect Americans under age 17 each year. Diarrhea results in 25 days of lost school, or work, for every 100 Americans, annually. Students are not the only ones to miss school due to infectious illnesses. Teachers miss even more school days than do students. One study showed that teachers lost an average of 5.3 days a year, in comparison to an annual average of 4.5 days loss for students.

So then, whether in the work place, the shopping environment, in school, at home, or while enjoying leisure activities, it is easy to appreciate the value of educating the public about not only the importance of proper hand washing but offering the public a way to make adhering to simple hygiene tasks, especially in the winter/flu season, more feasible. Learning proper hygiene, of course, should begin at an early age and children should be taught how to wash their hands effectively in their home. However, even some adults are not aware of the benefits of proper hand washing. A product that would encourage people, young and old, to wash their hands for the recommended amount of time, would be of great benefit to all people and in all situations where frequent and complete hand washing is required.

According to the Center for Disease Control (CDC) cleaning our hands is the single most important thing we can do to keep from getting sick and spreading illness to others. In other words, proper hand cleaning is the simplest and least expensive prevention against communicable illness. One study showed that proper hand cleaning four times a day can reduce gastrointestinal illness and related absences by more than 50 percent. The Soap and Detergent Association (SDA) suggests washing hands before a meal, after partaking in any activity that might bring your hands into contact with germs, such as after using the bathroom, when preparing food, before eating, after touching animals, whenever your hands are dirty, after being in public places or in public transportation vehicles, after coughing and sneezing, and when you or someone near you is ill. The SDA and the CDC recommend washing your hands with soap for at least 20 seconds to successfully remove germs and dirt.

The Soap and Detergent Association recommends that effective hand washing includes the following five steps.
1) Wet hands with warm running water prior to reaching for the soap, either in bar or liquid form.
2) Rub hands together to make a soapy lather. Do this away from running water, so the lather isn't washed away.
3) Wash both the front and the back of your hands, between your fingers, and under your nails. Continue washing for 15 seconds or more.
4) Rinse hands completely under warm running water.
5) Dry hands thoroughly with a clean towel or air dryer.

As simple and quick as this method for ensuring better health is, it is often difficult to get children and even many adults to wash their hands for the recommended 15 seconds. A frequent excuse offered is that the hand washer does not know with any accuracy when 15 seconds has elapsed. When the SDA conducted a Clean Hands Survey of 1013 American adults, 43 percent of the respondents said that they seldom or never washed their hands after coughing or sneezing. Nearly one-third of the people surveyed do not always wash their hands before eating, more than half of the respondents do not wash their hands long enough to effectively remove infectious agents and dirt, and 90 percent said that they always washed their hands after going to the bathroom. If, however, survey responses are compared with observational studies, there is a significant disparity between what people say and what they do. A study conducted by the American Society for Microbiology found that only about 60 percent of the people using the restroom washed their hands.

It is clear that what is still lacking in the art of personal hygiene is a means of encouraging both young and old to wash their hands for the recommended amount of time. There is a plethora of hand soaps on the market. None of these products, however, have successfully encouraged the public to wash their hands as recommended. There are bar soaps, liquid soaps, and powdered soaps that are all available in a wide variety of colors and fragrances. For children there are bar soaps in the shapes of animals and cartoon characters. There are soaps with creams to help keep hands smooth and there are soaps with anti-bacterial ingredients. The many anti-bacterial soap products on the market today attempt to ensure complete cleansing of hands, but the benefits of repeatedly washing one's hands with an anti-bacterial agent have been questioned.

Anti-bacterial soaps eliminate not only harmful infectious agents from the skin of one's hands, they also eliminate beneficial bacteria from the skin surface of one's hands. The elimination of the beneficial bacteria may result in dry hands. In fact, some people find that using anti-bacterial soaps have left the skin of their hands not only dry, but red, chapped, and in the more severe cases, cracked and bleeding. To effectively cleanse hands of germs, ordinary soap or a gentle detergent may be used, if one washes their hands for the recommended 15-20 seconds.

### SUMMARY

Accordingly, the present invention satisfies the current unmet need for an improved hand washing means that encourages effective 15-20 second hand washing. The invention, used several times each day, helps protect everyone's health in that proper hand washings spare family and friends, as well as others, from recurrent infections and disease.

The invention, as taught herein, offers a low cost and very easy to use means of solving the problem of ineffective hand washing. The invention comprises a hand washing composition (available in a powder, liquid, or solid form) that includes in the formulation an encapsulated, non-toxic, timed-release dye. During the hand washing routine, the non-toxic dye remains encapsulated for the required 15-20 seconds of washing. When 15-20 seconds have elapsed, the timed-release encapsulant releases a dye indicating that the hands have been washed for the recommended effective amount of time. The colored dye is easily and quickly rinsed off of the hands. Once all of the dye is completely removed by rinsing, the hand washer is assured that his or her hands have not only been washed successfully, but also been rinsed completely. In those cases where an anti-bacterial agent is recommended, it too should be used for the recommended 15-20 seconds, in order to be effective.

Accordingly, the present invention offers both the means and method to help the public achieve good hand hygiene regardless of whether a gentle hand washing product or an anti-bacterial product is used. Moreover, the present invention provides the means and method for cleaning and/or treating an article when the article needs to be cleaned and/or treated according to a timed schedule. For example, to parents' dismay, many children come home from school with an infection of head lice. Some treatments for head lice require an application of a medicated shampoo. These treatments generally require the formulation to be maintained on the head for a specified amount of time to be effective. Moreover because of the potency of these treatments, it is recommended that the treatment time not be extended more than the specified amount of time. Again, because of the potency of the product, thorough rinsing of the product from the head is strongly recommended. The present invention offers the means (a timed-release dye) to assure that such a product is kept in contact with the head being treated and means to assure that the product is rinsed off of the head completely (when all of the dye is rinsed off, so is the product).

The present invention offers all these benefits by providing for:
a timed-release cleansing and/or treatment formulation, comprising:
   a) a cleansing formula;
   b) an encapsulant, and
   c) a dye,
      wherein said dye is encapsulated by said encapsulant,
      wherein said encapsulated dye is contained in said cleansing formula, and
      wherein said encapsulant releases said dye after a predetermined amount of time of
      washing an article using said timed-release cleaning formulation.

In a preferred embodiment the encapsulant and the dye are water soluble.

In a more preferred embodiment the timed-release cleansing and/or treatment formulation is formulated for hand-washing use.

Furthermore, the timed-release cleansing and/or treatment formulation further comprises wherein the encapsulant releases the dye in the time recommended for thorough hand-washing.

Additionally, the timed-release cleansing and/or treatment formulation may be formulated with the addition of an encapsulated hand-cream and/or an encapsulated perfume.

In yet another embodiment, a method for making a timed-release cleansing and/or treatment formulation, is contemplated wherein the cleansing formula comprises the steps of:
a) providing a cleansing formula;
b) providing an encapsulant;
c) providing a dye;
d) encapsulating the dye by the encapsulant, and
e) containing the encapsulated dye in the cleansing formula,
   wherein the encapsulant releases said dye after a predetermined amount of time of washing using the timed-release cleaning formulation.

A method for the thorough cleaning of an article using a timed-release cleansing and/or treatment formulation is yet still further contemplated wherein the method comprises the steps of:
a) providing a cleansing formulation containing an encapsulated timed-released dye;
b) wetting an article to be cleaned with water;
c) applying the cleansing formulation to the wet article;
d) lathering the article with the cleansing formulation until the encapsulated timed-released dye is released, and
e) rinsing the lathered article thus provided for complete cleaning of the article.

Still other benefits and advantages of this invention will become apparent to those skilled in the art upon reading and understanding the following detailed specification and related drawings.

### BRIEF DESCRIPTION OF THE DRAWINGS

In order that these and other objects, features, and advantages of the present invention may be more fully comprehended and appreciated, the invention will now be described, by way of example, with reference to specific embodiments thereof which are illustrated in appended drawings wherein like reference characters indicate like parts throughout the several figures. It should be understood that these drawings only depict preferred embodiments of the present invention and are not therefore to be considered limiting in scope, thus, the invention will be described and explained with additional specificity and detail through the use of the accompanying drawings, in which:

FIG. 1 is a perspective view of the hand washing formulation means of this invention being applied to a pair of hands for washing in accordance with the principles of the present invention also showing an exploded view of the timed-release dye microencapsulates of the formulation.

FIG. 2 is a perspective view of the manner in which the hand washing formulation containing the timed-release dye microencapsulates is spread over one's hands following a recommended hand washing technique of lathering for 15-20 seconds, at which time the dye stored in the timed-release dye microencapsulates of the formulation, as shown in FIG. 1, is released indicating that proper hand washing time has been reached.

FIG. 3 is a perspective showing the hands shown in FIG. 2 being rinsed clean of the hand washing formulation and of the dye that was contained in the formation using a stream of water in accordance with the method of present invention.

FIG. 4 is a flow chart showing the steps providing for a method of making a timed-release encapsulated dye cleansing and/or treatment formulation according to the principles of the present invention.

FIG. 5 is a flow chart showing the steps providing for a method for thorough cleaning and/or treatment of an article according to the principles of the present invention.

### LIST OF REFERENCE NUMBERS AND PARTS OF THE INVENTION TO WHICH NUMBERS REFER

12 A container for containing a hand washing formulation of the present invention.
14a A right hand.
14b A left hand.
16 A hand washing formulation according to the present invention.
18 Part of a lathered hand showing the dye/lather mixture thoroughly rinsed off.
20 An encapsulate encapsulating a dye.
22 A dye within the encapsulation device.
24 Partially dissolved encapsulate in the act of time releasing dye 22.
30 The timed-released encapsulated dye intimately mixed with the cleansing lather.
32 Rinse water containing the dye/lather mixture.
40 Running water to rinse hands.
   Definitions
   Encapsulate, as used herein, in general refers to the coating that is applied to or around solid particles, liquid droplets, or gas bubbles with a thin film of coating or shell material. An encapsulate may also be referred to as an encapsulant.
   Microencapsulate, as used herein, refers to the coating of small (having diameters between 1 and 1000 μm) solid particles, liquid droplets, or gas bubbles with a thin film of coating or shell material. Although “microencapsulate” is the term used throughout the specification, its use is simply exemplary as Applicant contemplates the use of macroencapsulates as well as microencapsulates in the formulations of the present invention.
   Macroencapsulate, as used herein, refers to the coating of large (having diameters greater than 1000 μm) solid particles, liquid droplets, or gas bubbles with a thin film of coating or shell material. Macroencapsulates are also referred to as microgrannules.
   Timed-release, as used herein, refers to a mechanism that is used to release an encapsulated active agent after a certain time has elapsed. In this instance, the active agent may be one of, or a combination of, a dye, a perfume, a hand-cream, or the like that is present in an encapsulated form in a delivery formulation, wherein the encapsulate is programmed to release the active agent after a predetermined time, such as after a predetermined time of washing one hand's with a cleansing formulation containing time-releasing encapsulate coating a dye.

It should be understood that the drawings are not necessarily to scale. In certain instances, details which are not necessary for an understanding of the present invention or which render other details difficult to perceive may have been omitted.

### DETAILED DESCRIPTION

Referring now, with more particularity, to the drawings, it should be noted that the disclosed invention is disposed to embodiments in various sizes, shapes, and forms. Therefore, the embodiments described herein are provided with the understanding that the present disclosure is intended as illustrative and is not intended to limit the invention to the embodiments described herein.

The present invention is directed towards a novel cleaning formulation that is designed to indicate when cleaning, such as the cleaning of hands is complete. Poor or improper hand-washing is known to be a major cause of the spread of disease. Proper or thorough hand washing means lathering one's hands for at least 15-20 seconds, followed by a complete rinsing, and drying. This simple and effective procedure seems, however, difficult for people, young and old to follow. One reason why people do not cleanse their hands for the recommended amount of time, is that it is difficult for most people, especially children to know exactly when 15-20 seconds has elapsed.

The invention, as described, teaches the means and method that will help ensure proper and thorough hand cleaning. The invention introduces a cleaning formulation that indicates when, in this case, the recommended 15-20 seconds of hand washing has occurred by designing the cleaning formulation to contain an encapsulated water soluble dye, wherein the encapsulate is programmed to release the dye in the predetermined amount of time.

The water soluble dye that is easily rinsed may be of any desired color. Bright colors, of course appeal to children and will help to make proper hand washing fun for them. Along with the dye, a fragrance, an antibiotic, or a hand cream may be added to offer added benefits that will help to make proper hand washing more desirable for adults and children.

The presence of the dye serves not only to indicate that the lathering step of proper washing has been conducted for the recommended amount of time, but also helps to ensure that the hand washer also performs a complete hand rinsing. A hand washer will know when a rinse is complete, because the dye will no longer be visible.

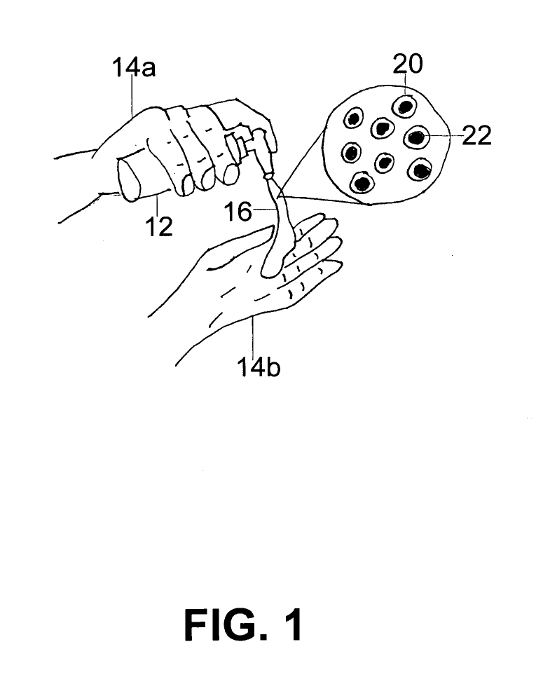

Now turning to the drawings, FIG. 1 shows pump-type container 12, held in user's right hand 14a, to dispense timed-release hand washing cleansing formulation 16 of the present invention onto left hand 14b. The timed-release cleansing formulation comprises a cleansing formulation that incorporates a timed-release encapsulated dye, where timed-release encapsulant is physically and/or chemically designed to release dye after a predetermined amount of time of washing.

FIG. 1 also shows an exploded view of the timed-release dye encapsulates of the formulation made in accordance with the principles of the present invention. In FIG. 1, which is an example of a favored embodiment, microencapsulates 20 encapsulate dye 22.

Encapsulates may be referred to as carriers, coatings, membranes, shells, or walls. The material being encapsulated may be referred to as an active agent, actives, core material, fill, internal phase (IP), nucleus, and payload. Encapsulated materials may be formulated as a single shell around a central core, a multinuclear encapsulated where a plurality of small domains of actives are distributed uniformly throughout a matrix of carrier, and a core being encased by a plurality of shells. The active may be a solid, liquid, or gas. Encapsulation is a mature field of technology. The various methods of encapsulating actives and the materials used to encapsulate are well-known to those in the art and need not be discussed in detail here. It suffices to say, that when, as in the present example, the timed-release cleansing formulation is a timed-released hand cleaning formulation, the encapsulate comprises a water soluble material that is designed to react (e.g., dissolve) in a predetermined time when in contact with water and under the effect of the friction of rubbing, as when one is in the process of lathering their hands. The encapsulant particles may be formulated as micro-particles that are invisible in the formulation or may be macro-particles that are clearly visible in the formulation and are used to provide consumer “value-added” appeal. The encapsulating material may be colored or it may be colorless and transparent to allow the color of the dye to be seen.

Dye technology is older and more developed than encapsulation technology and, thus, also does not need to be discussed in detail here. When, as in the present example, the timed-release cleansing formulation contains an encapsulated dye in a hand washing formulation, the dye comprises a water soluble, non-toxic dye that is easily rinsed from one's hands, especially after being incorporated into the lather formed by the hand washing composition.

Although, the example that is used for illustration purposes comprises a timed-release hand washing formulation, the formulation is also contemplated to be a treatment formulation that may or may not be part of a cleansing formulation. Examples of treatment formulations that are likely to be part of a cleansing formulation include medicated shampoo formulations, such as dandruff or lice treating shampoo. Additionally, cleansing formulations could include formulations for cleaning surfaces, such as a hard surface cleaning formulation, a soft surface cleaning formulation, or the like. Moreover, the formulation could be a treatment formulation that does not include a cleansing formulation. Such an example of a timed-release formulation that is not a hand cleaning formulation is paint or rust remover, a hair dye, or a myriad of other formulations that are applied to a surface for a specified period of time.

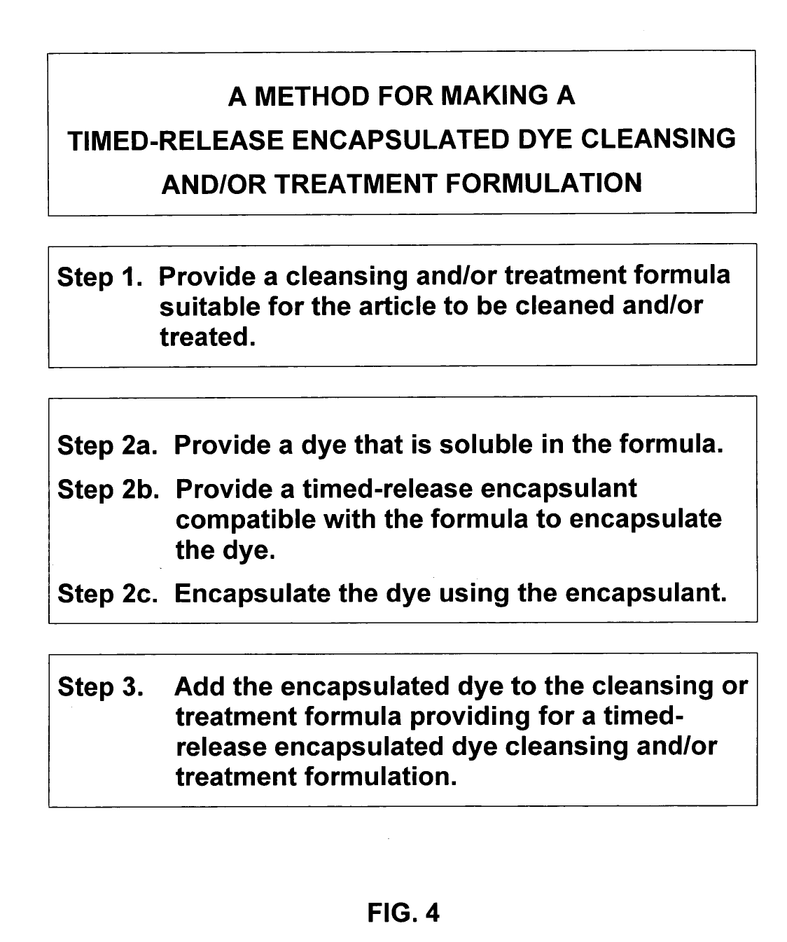

A method for making a timed-release cleansing and/or treatment formulation, as illustrated in FIG. 4, comprises the steps of providing a cleansing and/or a treatment formula to which is added a dye which has been encapsulated wherein the encapsulant is designed to release the dye after a predetermined amount of time of cleaning and/or treatment. The cleansing formulation may be soap, detergent, or alcohol based and it may, if desired, contain an antibiotic.

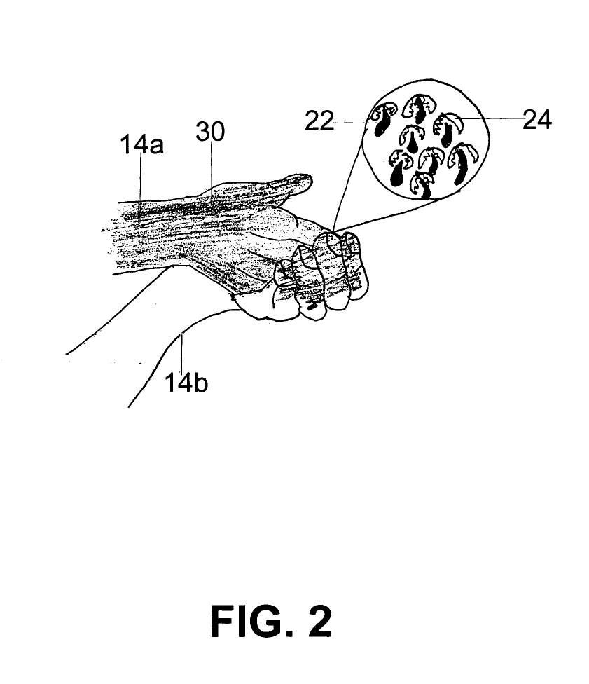

FIG. 2 is a perspective view showing the hand washing formulation containing the timed-release, now partially dissolved, encapsulates 24 in use following a recommended hand washing technique consisting of wetting, followed by lathering for 15-20 seconds, at which time encapsulated dye 22 stored in the timed-release dye microencapsulates of the formulation, as shown in FIG. 1, is released from the encapsulate indicating that proper lathering time has been reached. When the timed-release encapsulated dye 22 is released, it immediately is intermixed with the cleansing lather to form lather-dye mixture 30.

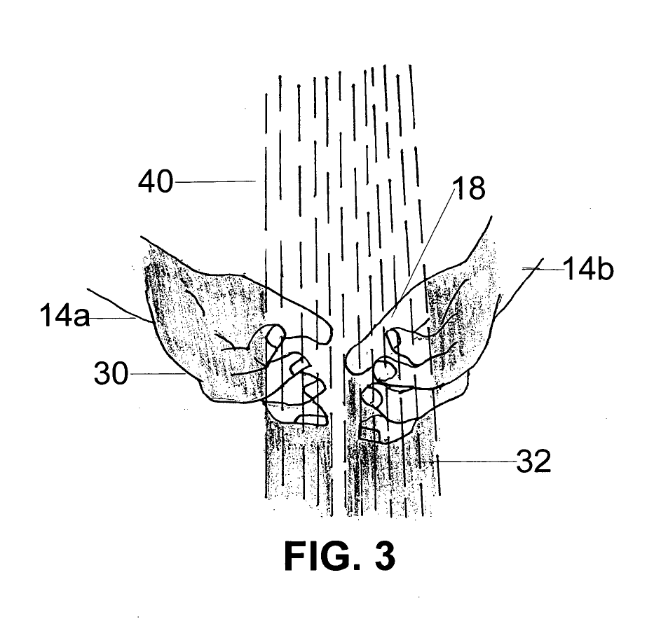

FIG. 3 illustrate the lather-dye covered hands, as shown FIG. 2, being rinsed clean of the hand washing/dye formulation using stream of water 40 in accordance with the method of present invention and in accordance with recommended thorough hand washing procedure. FIG. 3 illustrates a mid-point in the rinsing procedure where parts of the hands 18 have been rinsed clean as evidenced by the absence of the hand washing/dye mixture on that part of the hands. The water soluble dye hand washing formula 32 are easily and quickly rinsed off of the washed hands by the rinse water.

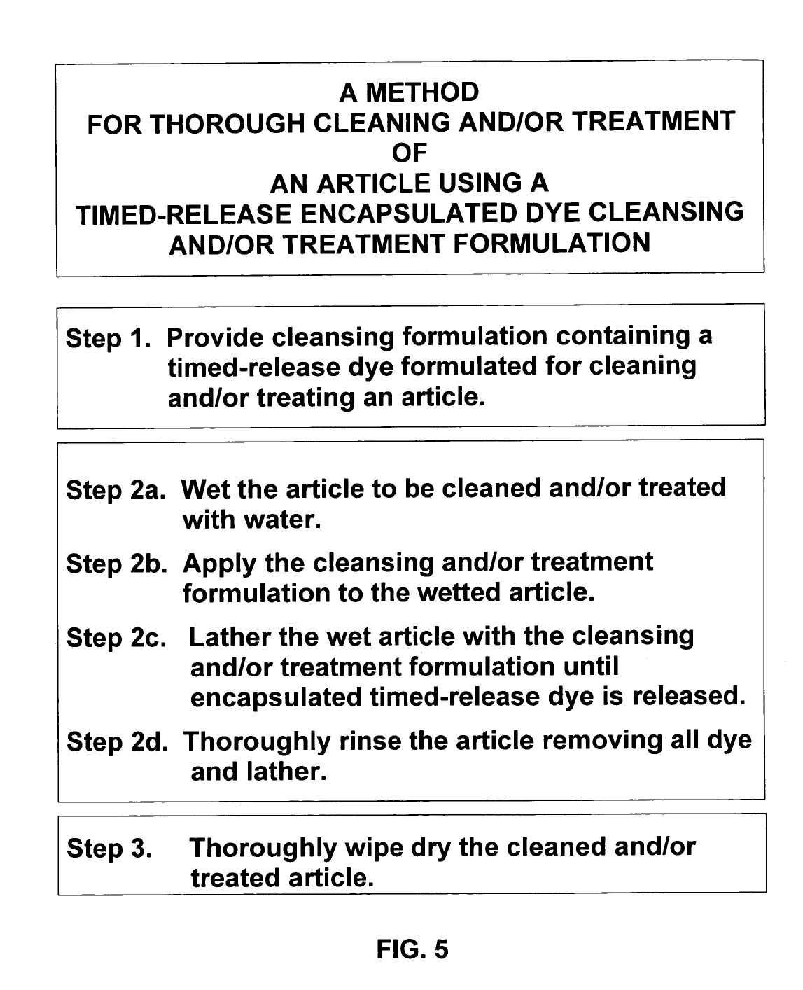

The steps that may be used to clean an article using the timed-release cleansing formulation of the present invention is illustrated in the flow diagram of FIG. 5. If the article to be cleaned is a pair of hands, the method's first step is to provide a user with a hand washing formulation containing timed-release encapsulated dye. The cleaning procedure is performed by wetting the hands with water. Next, the cleansing formulation is applied to the wetted hands. To perform a thorough hand washing as recommended, the wetted cleansing formulation should be worked into a lather for at least 15-20 seconds ensuring that the lather covers both front and back of the hands, between the fingers, and under the fingernails. When the recommended 15-20 seconds has elapsed, the encapsulated timed-release dye is released, due in part to the encapsulant dissolving and in part to the friction causing by the hands being rubbed together during the lathering process. The release of the dye indicates that the hands have been properly lathered and are ready for rinsing. Rinsing should be continued under running water until no more dye can be seen, thus ensuring that the detergent or soap based cleansing formulation is completely rinsed off the hands. The final step is to thoroughly dry the hands.

The time for the timed-release of the dye from its encapsulate can be adjusted as desired. For proper hand cleaning, the time for timed-release of the encapsulated dye would be from 15 to 20 minutes. If the cleansing formulation were a medicated shampoo, for example, the timed-release might be for a longer or shorter period of time.

The foregoing description, for purposes of explanation, uses specific and defined nomenclature to provide a thorough understanding of the invention. However, it will be apparent to one skilled in the art that the specific details are not required in order to practice the invention. Thus, the foregoing description of the specific embodiment is presented for purposes of illustration and description and is not intended to be exhaustive or to limit the invention to the precise form disclosed. Those skilled in the art will recognize that many changes may be made to the features, embodiments, and methods of making the embodiments of the invention described herein without departing from the spirit and scope of the invention. Furthermore, the present invention is not limited to the described methods, embodiments, features or combinations of features but include all the variations, methods, modifications, and combinations of features within the scope of the appended claims. The invention is limited only by the claims.

What is claimed is:

1. A timed-release cleansing and/or treatment formulation, comprising:
a) a cleansing and/or treatment formula;
b) an timed-release encapsulant, and
c) a dye,
wherein said dye is encapsulated by said encapsulant,
wherein said encapsulated dye is added to said formula providing for a cleansing and/or treatment formulation,
wherein said encapsulant releases said encapsulated dye after a predetermined amount of time of washing and/or treating using said formulation.

2. The timed-release cleansing and/or treatment formulation, as recited in claim 1, further comprising wherein said encapsulant is water soluble.

3. The timed-release cleansing and/or treatment formulation, as recited in claim 1, further comprising wherein said dye is water soluble.

4. The timed-release cleansing and/or treatment formulation, as recited in claim 1, further comprising wherein said formula is formulated for hand-washing.

5. The timed-release cleansing and/or treatment formulation, as recited in claim 4, further comprising wherein said encapsulant releases said dye in time recommended for thorough hand-washing.

6. The timed-release cleansing and/or treatment formulation, as recited in claim 1, further comprising wherein said formulation is optionally formulated with a hand-cream.

7. The timed-release cleansing and/or treatment formulation, as recited in claim 1, further comprising wherein said formulation is optionally formulated with a perfume.

8. A method for making a timed-release cleansing and/or treatment formulation, comprising the steps of:
a) providing a cleansing and/or treatment formula;
b) providing a timed-release encapsulant;
c) providing a dye;
d) encapsulating said dye by said encapsulant, and
e) adding said encapsulated dye to said formula providing for a cleansing and/or treatment formulation,
wherein said encapsulant releases said encapsulated dye after a predetermined amount of time of washing and/or treating an article using said formulation.

9. The timed-release cleansing and/or treatment formulation, as recited in claim 8, further comprising wherein said encapsulant is water soluble.

10. The timed-release cleansing and/or treatment formulation, as recited in claim 8, further comprising wherein said dye is water soluble.

11. The timed-release cleansing and/or treatment formulation, as recited in claim 8, further comprising wherein said cleansing formula is formulated for hand-washing.

12. The timed-release cleansing and/or treatment formulation, as recited in claim 11, further comprising wherein said encapsulant releases said dye in the time recommended for thorough hand-washing.

13. The timed-release cleansing and/or treatment formulation, as recited in claim 11, further comprising wherein said formulation is optionally formulated with an encapsulated hand-cream.

14. The timed-release cleansing and/or treatment formulation, as recited in claim 11, further comprising wherein said formulation is optionally formulated with an encapsulated perfume.

15. A method providing for a thorough cleaning and/or treatment of an article using a timed-release cleansing and/or treatment formulation, comprising the steps of:
a) providing a cleansing and/or treatment formulation containing a timed-released encapsulated dye;
b) wetting with water an article to be cleaned or treated;
c) applying said formulation to said wetted article;
d) lathering said article with said formulation until said timed-release encapsulant releases said encapsulated dye after a predetermined amount of time, and
e) rinsing said lathered article.

16. The timed-release cleansing and/or treatment formulation, as recited in claim 15, further comprising wherein said encapsulant is water soluble.

17. The timed-release cleansing and/or treatment formulation, as recited in claim 15, further comprising wherein said dye is water soluble.

18. The timed-release cleansing and/or treatment formulation, as recited in claim 15, wherein said article to be cleaned and/or treatment further comprises at least one hand.

19. The timed-release cleansing and/or treatment formulation, as recited in claim 18, further comprising wherein said formulation is formulated for hand-washing.

20. The timed-release cleansing and/or treatment formulation, as recited in claim 19, further comprising wherein said encapsulant releases said dye in the time recommended for thorough washing and/or treatment of said article.
